(19) United States
(12) Patent Application Publication
(10) Pub. No.: US 2021/0011005 A1
(43) Pub. Date: Jan. 14, 2021

(54) SYSTEMS AND METHODS FOR EVALUATING IMMUNE RESPONSE TO INFECTION

(71) Applicant: Beckman Coulter, Inc., Brea, CA (US)

(72) Inventors: Liliana TEJIDOR, Coral Gables, FL (US); Robert T. MAGARI, Cooper City, FL (US); Diana CAREAGA, Miami, FL (US); Mohamad HASAN, Miami, FL (US); Sanghyuk SHIN, Carlsbad, CA (US)

(21) Appl. No.: 16/925,943

(22) Filed: Jul. 10, 2020

Related U.S. Application Data
(60) Provisional application No. 62/873,806, filed on Jul. 12, 2019.

(51) Int. Cl. G01N 33/50; G01N 15/10; G01N 15/14

(52) U.S. Cl. CPC G01N 33/5091; G01N 2015/008; G01N 15/1429; G01N 15/1031

(57) ABSTRACT

Systems and methods for characterizing immune response to infection using cellular analysis, such as a hematological cellular analyzer. In some instances, the immune response may be characterized as normal or abnormal based on one or more blood cell population parameters. In some instances, abnormal characterization may be used to identify patients with sepsis or at elevated risk of developing sepsis.

### CROSS-REFERENCE TO RELATED APPLICATION

This is related to, and claims the benefit of, provisional patent application 62/873,806 titled “systems and methods for evaluating immune response to infection”, filed in the United States Patent Office on Jul. 12, 2019.

### BACKGROUND

Sepsis is a life-threatening organ dysfunction caused by a dysregulated host response to infection. Sepsis is a global healthcare crisis, affecting over 30 million people worldwide each year. The occurrence of sepsis is increasing at an annual rate of 1.5%, making it a significant global healthcare concern. Sepsis has a high mortality rate, killing more individuals than prostate cancer, breast cancer and HIV/AIDS combined.

In addition to the human toll, sepsis is costly to healthcare organizations. Sepsis-related costs—which may include longer hospital stays, ICU admissions, hospital readmissions, and extensive testing and patient monitoring—surpass $24 billion.

Sepsis is a syndrome defined by a set of signs and symptoms. Although sepsis is associated with infection, there is no single cause of sepsis, which can arise from bacterial, viral or fungal infections. Of course, not all infections result in sepsis, and the etiology of sepsis is not well characterized at this time. Further, there is no known biomarker unique to sepsis. Clinicians may rely on non-specific indicators such as fever, white blood cell counts (WBC), and altered mental state (AMS) to identify patients who might have sepsis. These tests are non-specific in that they are present in a variety of conditions other than sepsis, including some cases of non-septic infections, trauma, burns, cancers, etc. Diagnostic tests, including tests for procalcitonin (PCT) and C-Reactive Protein (CRP) are available but are not desirably specific or sensitive to sepsis. That is, available diagnostic tests both test positive for patients who are not septic and test negative for patients who are septic or who are developing sepsis, at undesirably high rates.

There remains a need for diagnostic tests which can help a clinician distinguish sepsis from other conditions, including flu, trauma, cancer, and non-septic inflammation.

### BRIEF SUMMARY

In some aspects, this disclosure relates to a system for evaluating variation in a cell population parameter. The system may comprise a flowcell with a flow of a liquid containing a plurality of cells through the flowcell. The system may comprise one or more sensors for detecting light scatter, light transmission, electrical impedance, RF conductivity, or combinations thereof as cells pass through the flowcell. The system may comprise a processor for calculating cell population parameters based on a plurality of measurements of individual cells of the same or related types. The processor may characterize an immune response to infection based at least in part on one or more of the cell population parameters.

According to a first aspect, some embodiments may provide a method for characterizing inflammatory response to infection. In some embodiments, such a method may comprise flowing a body fluid sample through a flowcell, irradiating a plurality of cells in the body fluid sample in the flowcell, measuring light scatter and direct current impedance from individual cells of the plurality of cells, identifying individual cells within the plurality of cells based at least in part on the light scatter measurements or the direct current impedance measurements, analyzing one or more cell population parameters, and characterizing an inflammatory response to infection based at least in part on the one or more cell population parameters. In some such embodiments, the one or more cell population parameters may be selected from the group consisting of MO_DC_SD, NE_DC_SD, NE_DC_MEAN, NNRBC_UMALS_SD, MO_ALL_SD, NE_NO, LY_PC, NNRBC_MALS_SD, WNOP, MO_DC_MEAN, WDOP, NNRBC_UMALS_MEAN, BA_PC, NNRBC_MALS_MEAN, BA_PC, NNRBC_MALS_MEAN, EGC_LMALS_MEAN, NNRBC_DC_SD, NNRBC_LMALS_SD, NNRBC_ALL_MEAN, and combinations thereof. In some embodiments, analysis of the cell population parameters may be based at least in part on the light scatter or direct current impedance measurements.

According to a second aspect, in some embodiments such as described in the context of the first aspect, the body fluid sample may be whole blood.

According to a third aspect, in some embodiments such as described in the context of any of the first two aspects, the cell population parameter may be analyzed for cells from the plurality of cells classified as NNRBC.

According to a fourth aspect, in some embodiments such as described in the context of any of the first through third aspects, each analyzed cell population parameter may be compared to a corresponding reference range.

According to a fifth aspect, in some embodiments such as described in the context of the fourth aspect, the inflammatory response to infection may be characterized as abnormal if at least one of the analyzed cell population parameters is outside its corresponding reference range.

According to a sixth aspect, in some embodiments such as described in the context of the fourth aspect, the inflammatory response to infection may be characterized as abnormal if all of the analyzed cell population parameters are outside their corresponding reference ranges.

According to a seventh aspect, in some embodiments such as described in the context of the fourth aspect, the method may comprise determining whether the distribution width of measure volumes for a population of monocytes (MDW) is within an MDW reference range.

According to an eighth aspect, in some embodiments such as described in the context of the seventh aspect, the inflammatory response to infection may be characterized as abnormal if at least one of the analyzed cell population parameters is outside its corresponding reference range and the distribution width of the volume of the monocytes is greater than 19 channels.

According to a ninth aspect, in some embodiments such as described in the context of the fourth aspect, the method may comprise determining whether a count of white blood cells (WBC) in the body fluid sample is within a normal reference range.

According to a tenth aspect, in some embodiments such as described in the context of the ninth aspect, the inflammatory response to infection may be characterized as abnormal if at least one of the analyzed cell population parameters is outside its corresponding reference range and the WBC is less than 4,000 cells/mm³ or greater than 12,000 cells/mm³.

According to an eleventh aspect, in some embodiments such as described in the context of the first aspect, the method may comprise performing a first comparison comparing the distribution width of measured volumes for a population of monocytes (MDW) within the body fluid sample with a MDW reference range. In some such embodiments, the method may comprise performing a second comparison comparing at least one of the analyzed cell population parameters to a corresponding reference range. In some such embodiments, the method may comprise performing a third comparison comparing a count of white blood cells (WBC) in the body fluid sample with a WBC reference range. In some such embodiments, the inflammatory response to infection may be characterized based on a combination of the first comparison, the second comparison, and the third comparison.

According to a twelfth aspect, in some embodiments such as described in the context of the eleventh aspect, the inflammatory response to infection may be characterized as abnormal if the at least one of the analyzed cell population parameters is outside its corresponding reference range, the MDW is outside the MDW reference range, and the WBC is outside the WBC reference range.

According to a thirteenth aspect, in some embodiments such as described in the context of the eleventh aspect, local decision rules are applied to characterize the inflammatory response to infection if the at least one of the analyzed cell population parameters, the MDW and the WBC are not all within or all outside of their respective reference ranges.

According to a fourteenth aspect, some embodiments may provide a system comprising a transducer module for measuring at least light scatter and direct current impedance caused by cells passing through the flowcell of the method of any of the first through thirteenth aspects. In some such embodiments, a system may be provided which comprises a processor configured with instructions stored on a non-transitory computer readable medium for performing the method of any of the first through thirteenth aspects.

According to a fifteenth aspect, in some embodiments such as described in the context of the fourteenth aspect, the transducer module may comprise means for measuring RF conductivity. In some such embodiments, the means for measuring RF conductivity may be operable to measure RF conductivity of cells passing through the flowcell of the method of any of the first through thirteenth aspects, and may also be operable to measure RF conductivity of cells passing through a second flowcell comprised by the transducer module.

### BRIEF DESCRIPTION OF THE DRAWINGS

FIG. 1 is a flowchart of an exemplary cellular analysis process in accordance with aspects of this disclosure.

FIG. 2 is a schematic depiction of an exemplary cellular analysis system in accordance with aspects of this disclosure.

FIG. 3 is an illustration of an exemplary transducer module and associated components in accordance with aspects of this disclosure.

FIG. 4 is a simplified block diagram of an exemplary module system in accordance with aspects of this disclosure.

FIG. 5 is an exemplary heatmap highlighting differentially expressed markers between sepsis and non-sepsis patients in accordance with aspects of this disclosure.

FIG. 6 is an AUC-ROC graph for selected biomarkers differentially expressed in sepsis patients in accordance with aspects of this disclosure.

FIG. 7 is a flowchart of exemplary possible algorithms in accordance with aspects of this disclosure.

### DETAILED DESCRIPTION

Prior efforts to provide an objective, diagnostic test for sepsis have included hematological cellular analysis. Abnormal White Blood Cell Counts (WBC) are often associated with infection, although they are not specific to sepsis. More recently, evaluation of other cell populations, such as immature granulocytes, has been proposed as a factor to consider in evaluating the likelihood that a patient has or is developing sepsis. Other proposals have included looking at cell population parameters, such as monocyte volume distribution width or neutrophil volume distribution width, as potential indicators of sepsis. However, the literature is replete with varied proposals that are either vague suggestions to look for changes in a particular cellular sub-population (e.g., neutrophils) without guidance as to what features or population parameters might be relevant, or highly specific suggestions to look at a particular cell population parameter without generalizable insight. As such, prior literature regarding the use of cell population parameters to evaluate or characterize an immune response to infection, and in particular to identify or predict sepsis, is unhelpful in rationalizing a research plan for identifying new cell population parameters of interest for these purposes.

A blood sample from a patient can be analyzed manually, e.g., by smearing blood on a slide and visually examining the slide. A manual operator can make counts of cells and identify cells by type, e.g., red blood cells, platelets, white blood cells, possibly by using visual aids to facilitate counting and/or sizing cells on the slide. However, it may be desirable to automate the analysis of a blood sample from a patient. Beyond the convenience of having an automated process, an automated or semi-automated cellular analysis system may be able, for example, to count a vastly larger number of cells in a blood sample, or to gather information about individual cells and/or cell populations that would be extremely challenging or impossible for a human to collect at a comparable sample size. These abilities are important to producing sufficient data points for cell population statistics, such as distribution width, that are robust based on sample size.

As used herein, “patient” refers to a person or other animal from whom a body fluid sample is obtained, and may apply to research subjects, outpatients (people or animals seen for a brief visit with a medical practitioner, typically less than 1 day in duration, for medical assessment or treatment), inpatients (people or animals admitted to a medical care facility for 1 day or more, including, without limitation, hospitals, hospice care, rehabilitation facilities, and the like), or others. In some aspects, a patient may be under the current care of a clinician, such as a doctor, physician's assistant, nurse, nurse practitioner, chiropractor, surgeon, dentist, or the like. In some aspects, a “patient” may donate a sample without receiving medical assessment or treatment from the donation.

Cellular analysis systems may use a variety of techniques to identify, count and/or characterize cells. For example, a cellular analysis system may use electrical impedance to determine the volume and quantity of cells passing through an interrogation zone in a flowcell. As another example, a cellular analysis system may use imaging technology to capture optical representations of the cell and analyze the optical representations (which might or might not be human-comprehensible or amenable to conversion to human-comprehensible images) to determine the size and quantity of cells in an interrogation zone, either in a quiescent system or in a flowcell. As yet another example, a cellular analysis system may use flow cytometry to irradiate cells passing through a flowcell and measure the transmission and/or scatter of the light as it passes through the cell. The light scatter may inherently distinguish different cells of different kinds, sizes or characteristics, or the cells may be prepared with markers, such as fluorescent markers, to facilitate the identification, quantification and/or characterization of the cells based on cellular features marked—or unmarked—by the marker. A cellular analysis system may use combinations of these and/or other techniques to count, identify, and/or characterize cells. For example, a cellular analysis system may use a combination of electrical impedance and light scatter to analyze cells in a blood sample. If a combination of techniques is used, the techniques may employ hardware set up in serial progression (e.g., the same sample or aliquot of a sample is passed through multiple, separate interrogation zones), or in parallel progression (e.g., different aliquots of a sample are passed at essentially the same time through multiple, separate interrogation zones), or two or more techniques may be employed at the essentially the same time (e.g., a flow cell may be equipped to measure both electrical impedance and light scatter from the same sample or aliquot of sample in the same flow cell at essentially the same time). In this regard, essentially the same time means that the processes are running in overlapping time intervals for the same sample or different aliquots of the same sample. It is not essential to the practice of the invention that the different techniques be coordinated to occur at precisely the same time or in time intervals of equal duration.

A sample for analysis may be any biological fluid which contains cells. The biological fluid may, for example, be blood. The sample may be whole blood, e.g., the blood which has not been processed or modified except for the possible addition of an anticoagulant to prevent the blood from clotting, which would complicate flowing the blood through a flowcell for analysis. The sample may be processed, e.g., by dilution, by concentration, by separation into components (such as plasma, serum, and cells); by pretreatment (e.g., with cytometry markers, with a lyse to rupture/remove certain cell types, with a stain to modify the appearance of one or more cells, etc.), with a sphering agent, or otherwise as may be helpful to prepare the sample for analysis. The blood may be human blood or non-human animal blood. In some circumstances, the sample may be from a non-blood body fluid, such as urine, synovial fluid, saliva, bile, cerebrospinal fluid, amniotic fluid, semen, mucus, sputum, lymph, aqueous humour, tears, vaginal secretions, pleural fluid, pericardial fluid, peritoneal fluid, and the like. As with blood, if non-blood body fluids are sampled, the non-blood body fluids may be processed, e.g., concentrated or the cells otherwise enriched, as by centrifugation, to achieve a desirable cellular concentration or to enrich or modify certain sub-populations of cells for analysis. A possible advantage of evaluating whole blood may be the relatively large number of cells available for analysis in a relatively small sample. A possible advantage of analyzing non-blood body fluids and/or processed blood may be pre-segregation of certain cells of interest and/or a reduction in the number of cells, because of differences in the types and number of cells that normally occur in different body fluids. A lower number of cells may be helpful, for example, for characterizing individual cells.

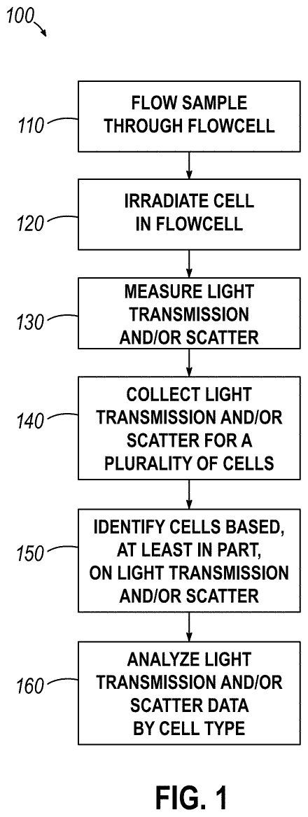

In some aspects, cells passing through a flowcell are analyzed using light scatter. As shown in FIG. 1, a method 100 for evaluating cell population variations may comprise flowing a sample through a flowcell 110. Cells within the sample flowing through the flowcell may be irradiated 120, as with visible light. The cellular analysis system may comprise one or more sensors which allow the analyzer to measure light transmission and/or scatter 130 as a cell is irradiated in the flowcell. The cellular analysis system may comprise a processor or means for communicating with a remote processor to collect the light transmission and/or scatter for a plurality of cells in the sample 140 as the cells flow through the flowcell. The processor may use an algorithm to identify cells based, at least in part, on light transmission and/or scatter 150. The processor, or a separate processor, may analyze the light transmission and/or scatter data for a particular cell or for a particular cell type population 160 (e.g., monocytes, neutrophils, red blood cells). The analysis could comprise, for example, calculating parameters, such as extrema, ranges, standard deviations, distribution widths, etc. for a particular measure, such as cell volume or light scatter, and/or for a particular cell type, such as monocytes, neutrophils, or NNRBC. For example, the cellular analysis system may calculate the standard deviation of light scatter, or of a particular angle of light scatter, such as upper median angle light scatter (UMALS), for cells identified as NNRBC. In some aspects, measurement 130 could involve alternative measurements of cell size and/or granularity, such as image analysis, electrical impedance, radiofrequency (RF) response, flow cytometry with or without markers, alone or in combination or sub-combinations, and with or without light transmission and/or light scatter measures.

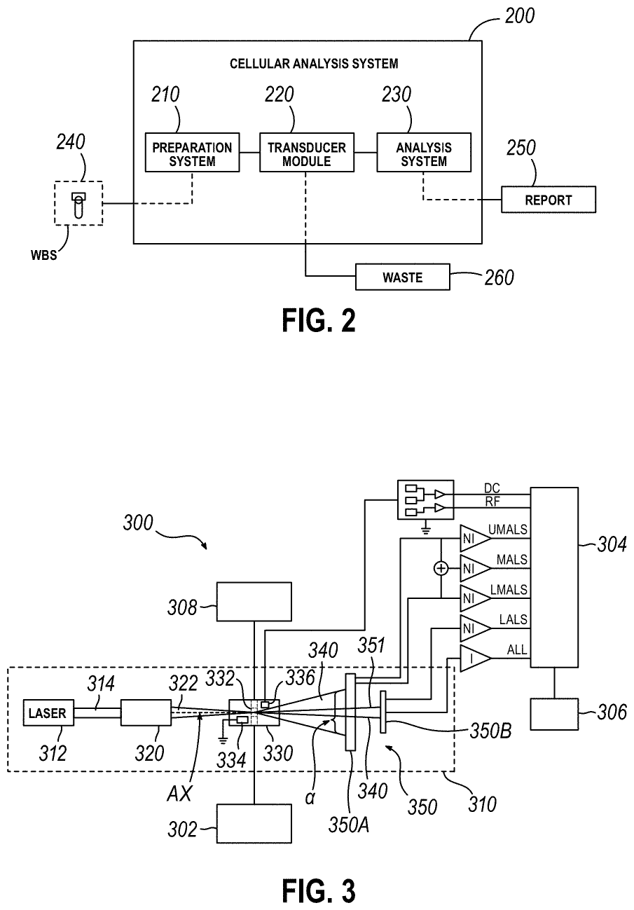

FIG. 2 schematically depicts a cellular analysis system 200. As shown here, system 200 includes a preparation system 210, a transducer module 220, and an analysis system 230. While system 200 is described generally with reference to three core system blocks (210, 220, and 230), the skilled artisan readily understands that system 200 may include other system components such as central control processor(s), display system(s), fluidic system(s), temperature control system(s), user-safety control system(s), and the like. In operation, a whole blood sample (WBS) 240 can be presented to the system 200 for analysis. In some instances, WBS 240 is aspirated into system 200. Exemplary aspiration techniques are known to the skilled artisan. After aspiration, WBS 240 can be delivered to a preparation system 210. Preparation system 210 receives WBS 240 and can perform operations involved with preparing WBS 240 for further measurement and analysis. For example, preparation system 210 may separate WBS 240 into one or more predefined aliquots for presentation to transducer module 220. In some aspects, preparation system 210 may make no changes to the composition of WBS 240. Alternately, preparation system 210 may include mixing chambers so that appropriate reagents may be added to one or more of the aliquots. For example, where an aliquot is to be tested for differentiation of white blood cell subset populations, a lysing reagent (e.g. ERYTHROLYSE, a red blood cell lysing buffer) may be added to the aliquot to break up and remove the RBCs. Preparation system 210 may also include temperature control components to control the temperature of the reagents and/or mixing chambers. Appropriate temperature controls can improve the consistency of the operations of preparation system 210, and may facilitate pre-treatment of cells in the sample, e.g., with fluorescent markers, stains, or lyse.

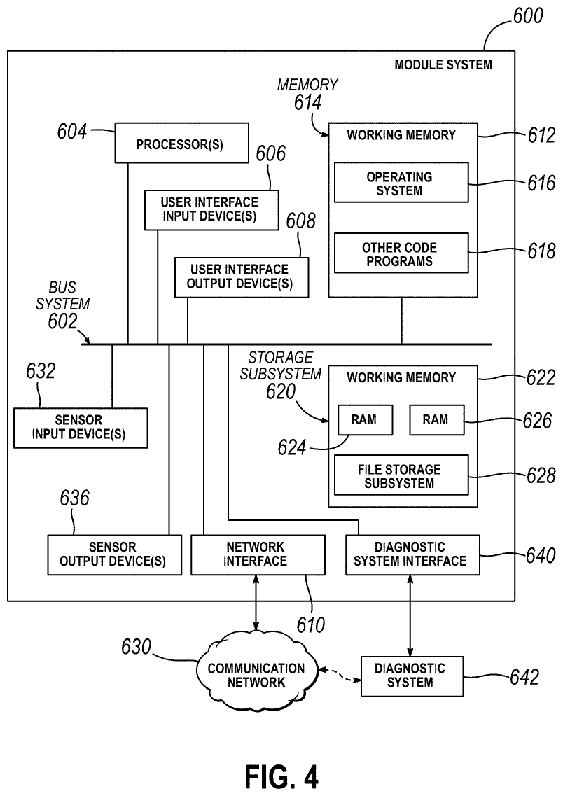

In some instances, one or more predefined aliquots can be transferred from preparation system 210 to transducer module 220. As described in further detail below, transducer module 220 can perform light transmission, and/or light scatter measurements of cells from the WBS 240 passing individually therethrough. Measured light propagation (e.g., light transmission, light scatter) parameters can be provided or transmitted to analysis system 230 for data processing. In some instances, analysis system 230 may include computer processing features and/or one or more modules or components such as those described herein with reference to the system depicted in FIG. 4 and described further below, which can evaluate the measured parameters, identify and enumerate at least one of the blood cellular constituents, and calculate cell population parameters for one or more cell populations within the aliquot. As shown here, cellular analysis system 200 may generate or output a report 250 containing measurements and/or calculated parameters for one or more cell populations within the aliquot, e.g., monocyte volume distribution width, neutrophil volume distribution width, a count or percentage of immature granulocytes, and/or a standard deviation of a UMALS measurement for NNRBC. In some instances, excess biological sample from transducer module 220 can be directed to an external (or alternatively internal) waste system 260. An exemplary cellular analysis system is a Beckman Coulter DxH hematology analyzer, which measures direct current impedance to determine cell volume, conductivity, and light scatter, for cytoplasmic granularity and nuclear structure.

Because there is no known biomarker specific to sepsis (e.g., in the sense that identifying a malarial parasite in a blood cell definitively indicates a malarial infection), there is currently no hematological analysis which can definitively diagnosis sepsis. However, identifying, enumerating and/or characterizing one or more cell populations in a patient samples may provide information which, in combination with clinical signs and symptoms and potentially with other tests or characterization studies, can reliably increase or decrease a clinical suspicion of sepsis or developing sepsis. Notably, because sepsis is a syndrome defined based on clinical symptoms, and because cell population changes may be observed before the clinical symptoms of sepsis, cell population data may help identify patients at high risk of developing sepsis, allowing for prophylactic treatment. This is advantageous because prophylactic treatment often involves the administration of antibiotic, antiviral and/or antifungal medications that pose challenges. For example, overuse of antibiotics in patients who are not septic or developing sepsis can contribute to the development of antimicrobial resistance. Further, some medications may have side effects or trigger adverse events that can be dangerous for a patient who is seriously ill or whose clinical state is declining. Accordingly, a test which can help a clinician develop an informed clinical treatment plan is valuable even if the test itself is not definitively diagnostic. Further, characterizing and/or enumerating cell populations that change during or in advance of a patient developing sepsis may be useful for non-diagnostic purposes, such as research into the etiology or progression of sepsis, or observing cellular responses to infection.

In some aspects, the analysis of a patient sample may cause a clinician to initiate and/or modify a treatment regimen. Treatment regimens may involve administration of one or more medications or therapeutic agents to an individual for the purposes of addressing the patient's condition. Any of a variety of therapeutic modalities can be used for treating an individual identified as having an abnormal immune response to infection, or having one or more abnormal cell population parameters as discussed herein. Exemplary therapies may include the administration of fluids, vasopressors, antibiotics, antifungals, antivirals, vitamins (including thiamine), minerals, steroids (including corticosteroids), and combinations thereof. In some instances, a patient may be subjected to more or less rigorous monitoring, including being admitted to a hospital for professional observation, based on the analysis of the patient sample.

FIG. 3 illustrates in more detail a transducer module and associated components in more detail. As shown here, system 300 includes a transducer module 310 having a light or irradiation source such as a laser 312 emitting a beam 314. The laser 312 can be, for example, a 635 nm, 5 mW, solid-state laser. In some instances, system 300 may include a focus-alignment system 320 that adjusts beam 314 such that a resulting beam 322 is focused and positioned at a cell interrogation zone 332 of a flow cell 330. In some instances, the flow cell 330 receives a sample aliquot from a preparation system 302. Various fluidic mechanisms and techniques can be employed for hydrodynamic focusing of the sample aliquot within flow cell 330.

In some instances, the aliquot generally flows through the cell interrogation zone 332 such that its constituents pass through the cell interrogation zone 332 one at a time. In some cases, a system 300 may include a cell interrogation zone or other feature of a transducer module or blood analysis instrument such as those described in U.S. Pat. Nos. 5,125,737; 6,228,652; 7,390,662; 8,094,299; 8,189,187; and 9,939,453, the contents of which are incorporated herein by references for all purposes. For example, a cell interrogation zone 332 may be defined by a square transverse cross-section measuring approximately 50×50 microns, and having a length (measured in the direction of flow) of approximately 65 microns. Flow cell 330 may include an electrode assembly having first and second electrodes 334, 336 for performing DC impedance and/or RF conductivity measurements of the cells passing through cell interrogation zone 332. Signals from electrodes 334, 336 can be transmitted to the analysis system 304. The electrode assembly can analyze volume and conductivity characteristics of the cells using low-frequency current and high-frequency current, respectively. For example, low-frequency DC impedance measurements can be used to analyze the volume of each individual cell passing through the cell interrogation zone. High-frequency RF current measurements can be used to determine the conductivity of cells passing through the cell interrogation zone. Because cell walls act as conductors to high frequency current, the high frequency current can be used to detect differences in the insulating properties of the cell components, as the current passes through the cell walls and through each cell interior. High frequency current can be used to characterize nuclear and granular constituents and the chemical composition of the cell interior.

The light source in FIG. 3 has been described as a laser, however, the light source may alternatively or additionally include a xenon lamp, an LED lamp, an incandescent lamp, or any other suitable source of light, including combinations of the same or different kinds of lamps (e.g., multiple LED lamps or at least one LED lamp and at least one xenon lamp). As shown in FIG. 3, for example, incoming beam 322 irradiates the cells passing through cell interrogation zone 332, resulting in light propagation within an angular range a (e.g. scatter, transmission) emanating from the zone 332. Exemplary systems are equipped with sensor assemblies that can detect light within one, two, three, four, five, or more angular ranges within the angular range a, including light associated with an extinction or axial light loss measure. As shown, light propagation 340 can be detected by a light detection assembly 350, optionally having a light scatter detector unit 350A and a light scatter and/or transmission detector unit 350B. In some instances, light scatter detector unit 350A includes a photoactive region or sensor zone for detecting and measuring upper median angle light scatter (UMALS), for example, light that is scattered or otherwise propagated at angles relative to a light beam axis within a range from about 20 to about 42 degrees. In some instances, UMALS corresponds to light propagated within an angular range from between about 20 to about 43 degrees, relative to the incoming beam axis which irradiates cells flowing through the interrogation zone. Light scatter detector unit 350A may also include a photoactive region or sensor zone for detecting and measuring lower median angle light scatter (LMALS), for example, light that is scattered or otherwise propagated at angles relative to a light beam axis within a range from about 10 to about 20 degrees. In some instances, LMALS corresponds to light propagated within an angular range from between about 9 to about 19 degrees, relative to the incoming beam axis which irradiates cells flowing through the interrogation zone.

A combination of UMALS and LMALS is defined as median angle light scatter (MALS), which may be light scatter or propagation at angles between about 9 degrees and about 43 degrees relative to the incoming beam axis which irradiates cells flowing through the interrogation zone. One of skill in the art will understand that these angles (and the other angles described herein) may vary somewhat based on the configuration of the interrogation, sensing and analysis systems.

As shown in FIG. 3, the light scatter detector unit 350A may include an opening 351 that allows low angle light scatter or propagation 340 to pass beyond light scatter detector unit 350A and thereby reach and be detected by light scatter and transmission detector unit 350B. According to some embodiments, light scatter and transmission detector unit 350B may include a photoactive region or sensor zone for detecting and measuring lower angle light scatter (LALS), for example, light that is scattered or propagated at angles relative to an irradiating light beam axis of less than about 5.1 degrees. In some instances, LALS corresponds to light propagated at an angle of less than about 9 degrees, relative to the incoming beam axis which irradiates cells flowing through the interrogation zone. In some instances, LALS corresponds to light propagated at an angle of less than about 10 degrees, relative to the incoming beam axis which irradiates cells flowing through the interrogation zone. In some instances, LALS corresponds to light propagated at an angle of about 1.9 degrees±0.5 degrees, relative to the incoming beam axis which irradiates cells flowing through the interrogation zone. In some instances, LALS corresponds to light propagated at an angle of about 3.0 degrees±0.5 degrees, relative to the incoming beam axis which irradiates cells flowing through the interrogation zone. In some instances, LALS corresponds to light propagated at an angle of about 3.7 degrees±0.5 degrees, relative to the incoming beam axis which irradiates cells flowing through the interrogation zone. In some instances, LALS corresponds to light propagated at an angle of about 5.1 degrees±0.5 degrees, relative to the incoming beam axis which irradiates cells flowing through the interrogation zone. In some instances, LALS corresponds to light propagated at an angle of about 7.0 degrees±0.5 degrees, relative to the incoming beam axis which irradiates cells flowing through the interrogation zone. In each instance, LALS may correspond to light propagated an angle of about 1.0 degrees or more. That is, LALs may correspond to light propagated at angles between about 1.0 degrees and about 1.9 degrees; between about 1.0 degrees and about 3.0 degrees; between about 1.0 degrees and about 3.7 degrees; between about 1.0 degrees and about 5.1 degrees, between about 1.0 degrees and about 7.0 degrees, between about 1.0 degrees and about 9.0 degrees; or between about 1.0 degrees and about 10.0 degrees.

According to some embodiments, light scatter and transmission detector unit 350B may include a photoactive region or sensor zone for detecting and measuring light transmitted axially through the cells, or propagated from the irradiated cells, at an angle of about 0 degrees relative to the incoming light beam axis. In some cases, the photoactive region or sensor zone may detect and measure light propagated axially from cells at angles of less than about 1 degree relative to the incoming light beam axis. In some cases, the photoactive region or sensor zone may detect and measure light propagated axially from cells at angles of less than about 0.5 degrees relative to the incoming light beam axis less. Such axially transmitted or propagated light measurements correspond to axial light loss (ALL or AL2). As noted in previously incorporated U.S. Pat. No. 7,390,662, when light interacts with a particle, some of the incident light changes direction through the scattering process (i.e., light scatter) and part of the light is absorbed by the particles. Both of these processes remove energy from the incident beam. When viewed along the incident axis of the beam, the light loss can be referred to as forward extinction or axial light loss. Additional aspects of axial light loss measurement techniques are described in U.S. Pat. No. 7,390,662 at column 5, line 58 to column 6, line 4.

As such, the cellular analysis system 300 provides means for obtaining light propagation measurements, including light scatter and/or light transmission, for light emanating from the irradiated cells of the biological sample at any of a variety of angles or within any of a variety of angular ranges, including ALL and multiple distinct light scatter or propagation angles. For example, light detection assembly 350, including appropriate circuitry and/or processing units, provides a means for detecting and measuring UMALS, LMALS, LALS, MALS, and ALL.

Wires or other transmission or connectivity mechanisms can transmit signals from the electrode assembly (e.g. electrodes 334, 336), light scatter detector unit 350A, and/or light scatter and transmission detector unit 350B to the analysis system 304 for processing. For example, measured DC impedance, RF conductivity, light transmission, and/or light scatter parameters can be provided or transmitted to the analysis system 304 for data processing. In some instances, analysis system 304 may include computer processing features and/or one or more modules or components such as those described herein with reference to the system depicted in FIG. 4, which can evaluate the measured parameters, identify and enumerate biological sample constituents, and correlate a subset of data characterizing elements of the biological sample with one or more features or parameters of interest. As shown here, cellular analysis system 300 may generate or output a report 306 presenting measurements made or parameters calculated for the sample, such as WBC, MDW, or UMALS mean for NNRBC. In some instances, excess biological sample from transducer module 310 can be directed to an external (or alternatively internal) waste system 308. In some instances, a cellular analysis system 300 may include one or more features of a transducer module or blood analysis instrument such as those described in previously incorporated U.S. Pat. Nos. 5,125,737; 6,228,652; 8,094,299; 8,189,187 and 9,939,453.

FIG. 4 is a simplified block diagram of an exemplary module system that broadly illustrates how individual system elements for a module system 600 may be implemented in a separated or more integrated manner. Module system 600 may be part of or in connectivity with a cellular analysis system 200. Module system 600 is well suited for producing data or receiving input related to cellular analysis. In some instances, module system 600 includes hardware elements that are electrically coupled via a bus subsystem 602, including one or more processors 604, one or more input devices 606 such as user interface input devices, and/or one or more output devices 608 such as user interface output devices. In some instances, system 600 includes a network interface 610, and/or a diagnostic system interface 640 that can receive signals from and/or transmit signals to a diagnostic system 642. In some instances, system 600 includes software elements, for example, shown here as being currently located within a working memory 612 of a memory 614, an operating system 616, and/or other code 618, such as a program configured to implement one or more aspects of the techniques disclosed herein. Memory 614 may be non-transitory and/or embodied in tangible media, such as hardware.

In some embodiments, module system 600 may include a storage subsystem 620 that can store the basic programming and data constructs that provide the functionality of the various techniques disclosed herein. For example, software modules implementing the functionality of method aspects, as described herein, may be stored in storage subsystem 620. These software modules may be executed by the one or more processors 604. In a distributed environment, the software modules may be stored on a plurality of computer systems and executed by processors of the plurality of computer systems. Storage subsystem 620 can include memory subsystem 622 and file storage subsystem 628. Memory subsystem 622 may include a number of memories including a main random access memory (RAM) 626 for storage of instructions and data during program execution and a read only memory (ROM) 624 in which fixed instructions are stored. File storage subsystem 628 can provide persistent (non-volatile) storage for program and data files, and may include tangible storage media which may optionally embody patient, treatment, assessment, or other data. File storage subsystem 628 may include a hard disk drive, a floppy disk drive along with associated removable media, a Compact Digital Read Only Memory (CD-ROM) drive, an optical drive, DVD, CD-R, CD RW, solid-state removable memory, other removable media cartridges or disks, and the like. One or more or all of the drives may be located at remote locations on other connected computers at other sites coupled to module system 600. In some instances, systems may include a computer-readable storage medium or other tangible storage medium that stores one or more sequences of instructions which, when executed by one or more processors, can cause the one or more processors to perform any aspect of the techniques or methods disclosed herein. One or more modules implementing the functionality of the techniques disclosed herein may be stored by file storage subsystem 628. In some embodiments, the software or code will provide protocol to allow the module system 600 to communicate with communication network 630. Optionally, such communications may include dial-up or internet connection communications.

System 600 can be configured to carry out various aspects of methods of the present disclosure. For example, processor component or module 604 can be a microprocessor control module configured to receive cellular parameter signals from a sensor input device or module 632, from a user interface input device or module 606, and/or from a diagnostic system 642, optionally via a diagnostic system interface 640 and/or a network interface 610 and a communication network 630. In some instances, sensor input device(s) may include or be part of a cellular analysis system that is equipped to obtain multiple light angle detection parameters, such as in a Beckman Coulter DxH™ hematology analyzer. In some instances, user interface input device(s) 606 and/or network interface 610 may be configured to receive cellular parameter signals generated by a cellular analysis system that is equipped to obtain multiple light angle detection parameters, such as a Beckman Coulter DxH™ Hematology Analyzer. In some instances, diagnostic system 642 may include or be part of a cellular analysis system that is equipped to obtain multiple light angle detection parameters, such as a Beckman Coulter DxH™ Hematology Analyzer.

Processor component or module 604 can also be configured to transmit cellular parameter signals, optionally processed according to any of the techniques disclosed herein or known to one of skill in the art, to sensor output device or module 636, to user interface output device or module 608, to network interface device or module 610, to diagnostic system interface 640, or any combination thereof. Each of the devices or modules according to embodiments of the present disclosure can include one or more software modules on a computer readable medium that is processed by a processor, or hardware modules, or any combination thereof. Any of a variety of commonly used platforms, such as Windows, MacIntosh, and Unix, along with any of a variety of commonly used programming languages, may be used to implement embodiments of the present disclosure.

User interface input devices 606 may include, for example, a touchpad, a keyboard, pointing devices such as a mouse, a trackball, a graphics tablet, a scanner, a joystick, a touchscreen incorporated into a display, audio input devices such as voice recognition systems, microphones, and other types of input devices. User input devices 606 may also download a computer executable code from a tangible storage media or from communication network 630, the code embodying any of the methods or aspects thereof disclosed herein. It will be appreciated that terminal software may be updated from time to time and downloaded to the terminal as appropriate. In general, use of the term “input device” is intended to include a variety of conventional and proprietary devices and ways to input information into module system 600.

User interface output devices 606 may include, for example, a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from module system 600 to a user. In some instances, a cellular analysis system may not directly include a user interface output device, instead transferring data to a network, computer processor, or computer-readable non-transitory storage medium, with data display for a human user occurring in connection with that device or with devices to which the data from the cellular analysis system is further transferred after the initial transfer. If data is transferred from the analyzer without display, the data transferred may be raw sensor data or processed data or a combination of raw and processed data.

Bus subsystem 602 provides a mechanism for letting the various components and subsystems of module system 600 communicate with each other as intended or desired. The various subsystems and components of module system 600 need not be at the same physical location but may be distributed at various locations within a distributed network. Although bus subsystem 602 is shown schematically as a single bus, alternate embodiments of the bus subsystem may utilize multiple busses.

Network interface 610 can provide an interface to an outside network 630 or other devices. Outside communication network 630 can be configured to effect communications as needed or desired with other systems. It can thus receive an electronic packet from module system 600 and transmit any information as needed or desired back to module system 600. As depicted here, communication network 630 and/or diagnostic system interface 642 may transmit information to or receive information from a diagnostic system 642 that is equipped to obtain multiple light angle detection parameters, such as such as a Beckman Coulter DxH™ Cellular Analysis System. As non-limiting examples, outside communication network 630 may be used to transmit data between a cellular analysis system and a research database, a laboratory information system (LIS), an electronic medical record (EMR), and the like. In some instances, the communication may be one-way, with information flowing from the cellular analysis system to other systems. In some instances, the communication may be one-way with information (such as orders for specific measurements to be made or population parameters to be calculated) flowing from an external system, which may be remote or physically proximate to the cellular analysis system, to the cellular analysis system. In some instances, the communication may be two-way. In some instances, the information communicated to the cellular analysis system by an external system may include patient information useful in evaluating the significance of cellular measurements. For example, some reference ranges for hematology parameters may differ for pediatric populations or specific patient sub-populations relative to a general adult population, and the cellular analysis system may consider patient information when determining whether to flag analytical results for further review.

In addition to providing such infrastructure communications links internal to the system, the communications network system 630 may also provide a connection to other networks such as the internet and may comprise a wired, wireless, modem, and/or other type of interfacing connection.

It will be apparent to the skilled artisan that substantial variations may be used in accordance with specific requirements. For example, customized hardware might also be used and/or particular elements might be implemented in hardware, software (including portable software, such as applets), firmware, or combinations thereof. Further, connection to other computing devices such as network input/output devices may be employed. Module terminal system 600 itself can be of varying types including a computer terminal, a personal computer, a portable computer, a workstation, a network computer, or any other data processing system. Due to the ever-changing nature of computers and networks, the description of module system 600 depicted in FIG. 4 is intended only as a specific example for purposes of illustrating one or more embodiments of the present disclosure. Many other configurations of module system 600 are possible having more or less components than the module system depicted in FIG. 4. Any of the modules or components of module system 600, or any combinations of such modules or components, can be coupled with, or integrated into, or otherwise configured to be in connectivity with, any of the cellular analysis system embodiments disclosed herein. Relatedly, any of the hardware and software components discussed above can be integrated with or configured to interface with other medical assessment or treatment systems used at other locations.

In some embodiments, the module system 600 can be configured to receive one or more cellular analysis parameters of a patient at an input module. Cellular analysis parameter data can be transmitted to an assessment module where raw sensor data or partially analyzed sensor data is further processed and/or evaluated in conjunction with additional information, possibly including prior laboratory results for the same patient; laboratory results from other types of analyzers or laboratory analyses; non-laboratory data about the patient, such as patient complaints, diagnostic history, vitals, or physical examination findings, or combinations thereof. The cellular analysis, such as WBC, MDW, NNRBC UMALS mean, and other cell population parameters can be output to a system user via an output module. In some cases, the module system 600 can determine an initial treatment or induction protocol for the patient, or an adjusted treatment protocol, based on one or more cellular analysis parameters and/or the predicted sepsis status, for example by using a treatment module. The treatment can be output to a system user via an output module. Optionally, certain aspects of the treatment can be determined by an output device, and transmitted to a treatment system or a sub-device of a treatment system. Any of a variety of data related to the patient can be input into the module system, including age, weight, sex, treatment history, medical history, and the like. Parameters of treatment regimens or diagnostic evaluations can be determined based on such data.

Analysis system 304 of transducer module 300 or other code programs 618 of module system 600 or both may comprise one or more algorithms for processing sensor data generated by transducer module 300. The one or more algorithms may process the sensor data to identify and count cells. For example, individual cells may be identifiable based on light scatter and/or light transmission data that provides an indication of the size and certain surface properties of the cells. As another example, electrical impedance may provide an indication of the size of the cell. Radiofrequency conductivity may provide an indication of cellular constituents that may be useful in distinguishing granulocytes and nucleated versus non-nucleated cells. Markers, stains, image analysis or other measurement techniques may be used to identify cells as, for example, NNRBC, WBC, monocytes, neutrophils, and the like. An algorithm may count the number of signals consistent with a given cell type during an interrogation period, which may be defined by time through a flowcell or imaging time, or may be defined by a volume of body fluid examined, or both.

In some instances, the signals produced are continuous or ordinal values, and the magnitude or other properties of the signals may be further analyzed. For example, higher electrical impedance values typically indicate a larger cell, and may be useful for identifying a particular cell. Electrical impedance values may also correlate to cell volume, and therefore the magnitude of the signals across many cells in the sample may also convey useful information about that sub-population of cells. For example, electrical impedance values may help identify monocytes and distribution features of the volumes for the monocytes as a sub-population of cells may convey information about immune response to an infection.

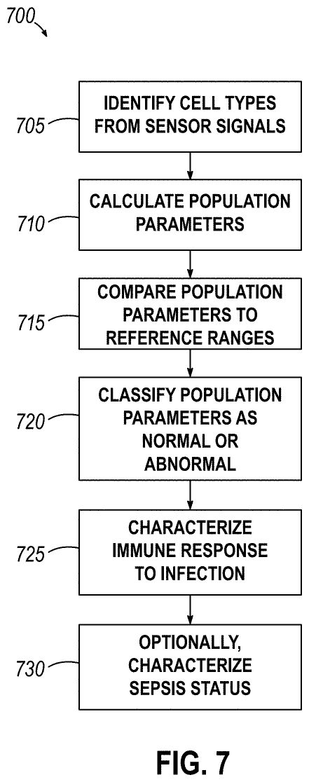

FIG. 7 is a flowchart for exemplary algorithms of potential use in practice of this disclosure. As shown, a single algorithm 700 encompasses all of the illustrated acts; however, different algorithms or even different software could be used, and, in particular, various acts could be performed by different algorithms or different software that might reside on or be processed by different hardware. Algorithm 700 may identify cell types from sensor signals 705. Algorithm 700 may calculate one or more cell population parameters 710. Algorithm 700 may compare the calculated cell population parameters to a reference range for each of the cell population parameters 715. Algorithm 700 may classify the cell population parameters as normal or abnormal in relation to their respective reference ranges 720. Based at least in part on the classification of the cell population parameters as normal or abnormal, algorithm 700 may characterize an immune response to an infection as normal or abnormal 725. Based at least in part on the classification of the cell population parameters as normal or abnormal, algorithm 700 may characterize the sepsis status of a patient 730. The sepsis status may be binary, e.g., yes or no. The sepsis status may be presented as a probability. The sepsis status may be presented as a qualitative flag, e.g., an indication that one or more cell population parameters are consistent with sepsis. The sepsis status may be presented as a risk level, e.g., low, moderate, or high risk of sepsis.

Algorithm 700 may use a single cell population parameter to characterize the immune response to infection and/or a sepsis status. Alternately, algorithm 700 may use two or more cell population parameters to characterize the immune response to infection and/or a sepsis status. In some aspects, algorithm 700 may use as many as 26 cell population parameters to characterize the immune response to infection and/or a sepsis status. For any number of cell population parameters considered, if all of the relevant cell population parameters are normal based on their respective reference ranges, for this purpose the immune response to infection is normal, and the patient is not identified as septic. If one or more of NNRBC-UMALS-SD, MDW and WBC are abnormal in relation to their respective reference ranges, then for this purpose the results as a whole are abnormal, and the algorithm may apply a global or local decision rule. A global decision rule is a threshold or operation applied uniformly to all data processed by algorithm 700. In contrast, local decision rules may be permitted to allow different institutions or different practitioners to establish different rules for identifying a patient as having an abnormal immune response to infection, or for identifying a patient as being septic or at elevated risk of developing sepsis. Local decision rules allow institutions to adapt the specificity (ability to inclusively identify most or all cases of possible sepsis) and sensitivity (ability to exclude most or all non-sepsis cases) of the algorithm, to reduce false negatives or false positives, respectively. In most or all cases, it is contemplated that if all of the relevant cell population parameters are abnormal, the results would be flagged as abnormal, and, if the results are used to identify sepsis, the patient would be identified as having sepsis or an elevated risk of developing sepsis. If the results are not all-normal or all-abnormal, the decision rules would determine whether to flag the results as a whole as abnormal with respect to immune response to infection and/or sepsis status. In some instances, the decision rules may weight different cell population parameters based on their observed correlation to sepsis status in prior clinical cases (e.g., in clinical trials). The results as a whole (e.g., across multiple relevant cell population parameters of interest) may be flagged as abnormal if the weighted majority or supermajority of relevant cell population parameters are abnormal. An observed correlation may be based on correlations of individual cell population parameters to sepsis status, or a multivariate model based on multiple cell population parameters. The results as a whole may be flagged as abnormal if any relevant cell population parameter is abnormal. The results as a whole may be flagged as abnormal if specified relevant cell population parameters are abnormal, even if others are normal. For example, the results as a whole may be flagged as abnormal if an NNRBC UMALS measure (such as mean or standard deviation), MDW and WBC are abnormal, even if other cell population parameters relevant to immune response or sepsis are normal. That is, selected cell population parameters or combinations of cell population parameters may be sufficient to treat the results as a whole as abnormal. The results as a whole may be flagged as abnormal only if specified relevant cell population parameters or combinations of cell population parameters are abnormal, even if others are normal. That is, selected cell population parameters may be necessary to treat the results as a whole as abnormal. For example, the results as a whole may be considered normal if WBC is normal, even if MDW, NNRBC UMALS measures, or others are abnormal.

If the overall results are identified by algorithm 700 as abnormal, this may be presented as a separate analytical result (e.g., Sepsis Indicated? Yes/No or Immune Response Measures: Normal/Abnormal), or may be presented as a flag to invite review by laboratory personnel and/or a clinician (e.g., text or other symbols or indicators in a report indicating that results indicate abnormal immune response to infection and/or indicate possible sepsis). Of course, in some instances, algorithm 700 may not apply any decision rules, deferring to laboratory and/or clinical personnel to interpret the results of the cellular analysis.

As noted above, a relatively new use of cellular analysis is the evaluation of the likelihood that a patient has or is at elevated risk (relative to a healthy person of similar age) of developing sepsis in the near-term (1 week or less). Such evaluation has so far centered on white blood cells or sub-populations of white blood cells, such as monocytes or immature granulocytes. However, different measures of white blood cells and measures of different white blood cell sub-populations (e.g., monocytes, lymphocytes, eosinophils, basophils, neutrophils, granulocytes, immature granulocytes, or combinations thereof) are more indicative of dysfunctional immune response to infection and/or sepsis than others. Further, the inventors have surprisingly found that there may be measurable distinctions in heterogenous populations of circulating blood cells, such as NNRBC, when a patient has or may be developing a dysfunctional immune response to an infection, or sepsis.

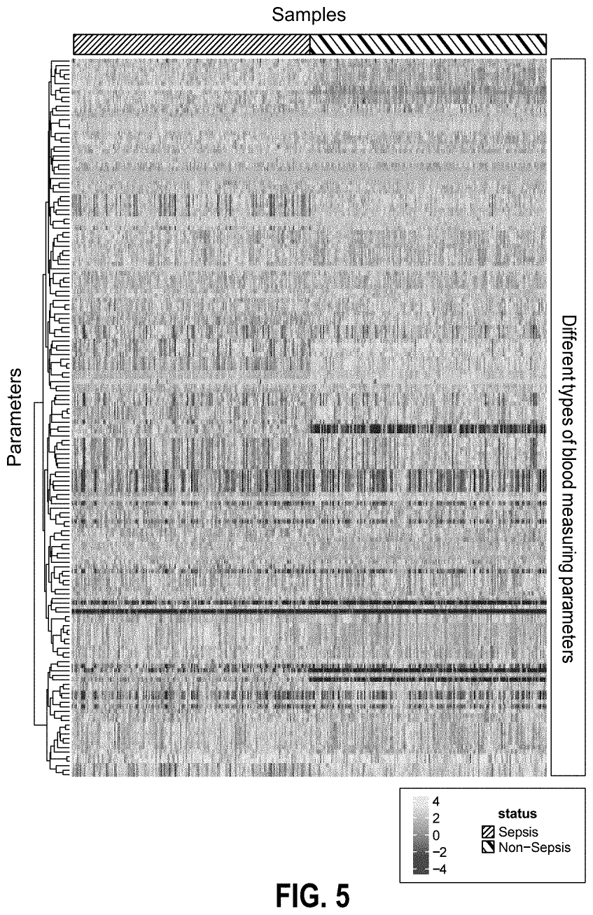

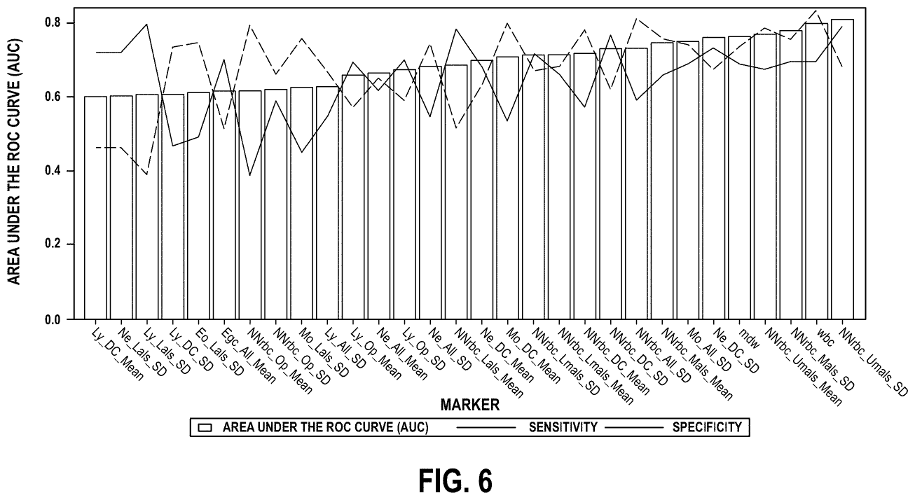

FIG. 5 is a heatmap highlighting differentially expressed markers between sepsis and non-sepsis patients. This analysis, as with FIG. 6, comes from data collected in a pivotal clinical trial involving adult patients, 18-89 yrs., with complete blood count with differential performed upon presentation to the emergency department (ED), and who remained hospitalized for at least 12 hours. A total of 2,158 subjects were enrolled and categorized per Sepsis-2 criteria: controls (n=1,088), systemic inflammatory response syndrome (SIRS) (n=441), infection (n=244), sepsis (n=385); and Sepsis-3 criteria: control (n=1,529), infection (n=386), sepsis (n=243). All 385 sepsis cases and randomly selected 385 non-sepsis cases were used for the analysis. Each column (line) in the Heatmap represents a sample, and samples were grouped into sepsis and non-sepsis patients. Each row represents a parameter. Rows were further grouped by clusters and differentiated by colors and dendrogram. Each gradation represents a cluster group, and the height of the dendrogram represents the negative-strength of the relationship between the markers. All values were standardized by markers to show the relative importance of the values or deviation of a value from the corresponding mean. The gradation represents the low-to-high magnitude of the values, and the black represents the missing value of the corresponding markers. Heatmap shows that many of the markers are highly differentiated between the sepsis and non-sepsis. To recognize significantly differentiated markers and strength of the significance, the following statistical analyses were performed.

Two types of statistical analyses were performed to explore promising sepsis markers further. First, Area Under Curve (AUC) and sensitivity and specificity were calculated for each marker and showed top markers with the highest AUC, as shown in FIG. 6. For each marker, the AUC was calculated using one predictor logistic regression model; and the cutoff and the corresponding sensitivity and specificity were calculated using Youden's J statistic. Youden's J statistic (also called Youden's index) is a single statistic that captures the performance of a dichotomous diagnostic test.

Second, covariate weighted multiple-hypothesis was performed to identify significant markers that were differentially expressed between sepsis and non-sepsis. Since the markers are highly correlated, multiple-hypothesis was essential to asses a set of statistical inferences/tests, simultaneously. Assessing multiple inferences can induce erroneous conclusion by chance. Therefore, a stricter significance threshold for individual comparisons is used to compensate for the number of inferences being made. Covariate weighting is one of the best ways of adjusting the significance threshold, in which a covariate (statistically independent of the test) is obtained to compute weight and consequently adjust original p-value dividing by the weight then compare against a given significance level. For this analysis, data were transformed to log-scale due to the skewness, then calculated the p-value using t-statistic to test the difference between sepsis and non-sepsis (Table 1). The weight was calculated using covariate rank weighting method where the standard deviation of each marker was used as covariate.

TABLE 1
Twenty statistically significant markers.

| | | | Adjusted | |
|---|---|---|---|---|
| Markers | P-value | Weight | P-value | AUC |
| MDW | 1.42E−18 | 0.81 | 2.44E−16 | 0.79 |
| (Diff_MDW_Value) | | | | |
| Mo_DC_SD | 1.09E−11 | 0.80 | 1.85E−09 | 0.78 |
| Ne_DC_SD | 0.000131 | 0.70 | 0.0258 | 0.77 |
| Ne_DC_Mean | 0.000247 | 1.86 | 0.0181 | 0.75 |
| NNrbc_Umals_SD | 1.62E−19 | 1.11 | 2.00E−17 | 0.75 |
| Mo_All_SD | 7.94E−06 | 0.52 | 0.00208 | 0.75 |
| WBC | 6.18E−18 | 0.43 | 1.98E−15 | 0.75 |
| (Pres_SIRS_WBC_Value) | | | | |
| Ne_No | 6.65E−18 | 0.24 | 3.83E−15 | 0.75 |
| Ly_Pc | 1.44E−06 | 0.59 | 0.000334 | 0.74 |
| NNrbc_Mals_SD | 2.60E−13 | 1.04 | 3.44E−11 | 0.73 |
| Wnop | 4.23E−15 | 0.32 | 1.81E−12 | 0.73 |
| Mo_DC_Mean | 0.000696 | 1.97 | 0.0486 | 0.72 |
| Wdop | 2.82E−12 | 0.33 | 1.19E−09 | 0.71 |
| NNrbc_Umals_Mean | 1.85E−06 | 2.04 | 0.000124 | 0.70 |
| Ba_Pc | 7.04E−06 | 0.12 | 0.00827 | 0.70 |
| NNrbc_Mals_Mean | 9.64E−06 | 2.07 | 0.000658 | 0.69 |
| Egc_Lmals_Mean | 2.08E−08 | 1.69 | 1.71E−06 | 0.68 |
| NNrbc_DC_SD | 2.87E−07 | 1.14 | 3.44E−05 | 0.68 |
| NNrbc_Lmals_SD | 9.42E−07 | 1.03 | 0.000126 | 0.66 |
| NNrbc_All_Mean | 0.000257 | 2.01 | 0.017 | 0.64 |

All = Axial Light Loss
Ba = Basophil
DC = Direct Current
EGC = Early Granulated Cell
Lmals = Lower Median Angle Light Scatter
Ly = Lymphocyte
Mals = Median Angle Light Scatter
Mo = Monocyte
Ne = Neutrophil
NNrbc = Not Nucleated Red Blood Cell (all population of WBC (Ly, Mo, Ne, Eo, Ba))
SD = Standard Deviation
Umals = Upper Median Angle Light Scatter
WBC = White Blood Cell

NNRBC_MALS_MEAN is the mean of MALS light scatter measures for cells identified as non-nucleated red blood cells (NNRBC). NNRBC_UMALS_MEAN is the mean of UMALS light scatter measures for cells identified as NNRBC. NNRBC_ALL_MEAN is the mean of ALL light transmission measures for cells identified as NNRBC. MO_DC_MEAN is the mean of direct current measurements for cells identified as monocytes. NE_DC_MEAN is the mean of direct current measures for cells identified as neutrophils. EGC_LMALS_MEAN is the mean of LMALS light scatter measures for cells identified as early granulocytic cells. NNRBC_DC_SD is the standard deviation of direct current measures for cells identified as NNRBC. NNRBC_UMALS_SD is the standard deviation of UMALS light scatter measures for cells identified as NNRBC. NNRBC_MALS_SD is the standard deviation of MALS light scatter measures for cells identified as NNRBC. NNRBC_LMALS_SD is the standard deviation of LMALS light scatter measures for cells identified as NNRBC. MDW is the distribution width of volume measures for cells identified as monocytes. MO_DC_SD is the standard deviation of direct current measurements for cells identified as monocytes. NE_DC_SD is the standard deviation of direct current measures for cells identified as neutrophils. LY_PC is the percentage of the WBC cells identified as lymphocytes. MO_ALL_SD is the standard deviation of ALL light transmission measures for cells identified as monocytes. WBC is a count of white blood cells. WDOP is leukocyte estimate (corrected) from the DIFF optical channel. WNOP is leukocyte estimate (corrected) from NRBC optical channel. NE_NO is the count of the WBC cells identified as neutrophils. BA_PC is the percentage of WBC cells identified as basophil.

The best of the inventors' knowledge, no prior study has looked at light scatter parameter changes for a heterogeneous cell population of circulating cells from a septic population, such as NNRBC, compared to controls. Prior studies have used hematological analyzers to look at light scatter changes in specific cell types during sepsis such as in specific lymphocyte, monocyte, or neutrophil cell populations (reviewed in Zonneveld R, Molema G, Plotz FB: Analyzing neutrophil morphology, mechanics, and motility in sepsis: options and challenges for novel bedside technologies. Crit Care Med 2016; 44: 218-228). With regard to cell surface granulation, no hypothesis-driven study has demonstrated a specific correlation between sepsis and cell surface granulation in any cell type.

It is well documented that sepsis causes a number of changes in circulating cells. Without wishing to be bound by theory, changes in membrane protein and lipid composition, changes in Na/Cl pump concentration, changes in ratios of cell types, and changes in the activation state of immune cells could be an underlying cause of a cellular granularity change that could impact light scattering. Any of these underlying biological mechanisms or combinations thereof could drive the observed light scatter differences in the NNRBC parameter. Nonetheless, obtaining the light scatter measurement and calculating particular cell population parameters, such as NNRBC-UMALS-SD, involve processes that would not occur in nature. The inventors have no indication that human-conducted visual examination of NNRBC granularity, e.g., via review of blood smear slides, would be useful in distinguishing septic and non-septic patients. Granularity can be assessed via blood smear review, but it is subjective and not standardized. Standard deviations cannot be visually assessed. In this study, mean NNRBC UMALS measurement was not as effective in identifying septic patients as NNRBC-UMALS-SD, suggesting that a human impression or estimate of granularity across a relatively small sample of cells would be unreliable for this purpose.

One of skill in the art will appreciate that the reference ranges and thresholds for assessing normality or abnormality for any given cell population parameter will vary based on the methods used to measure or observe the cells, as well as the specific hardware (e.g., light source, sensing hardware) used to make the measurements. Not only the reference range but also the unit of measure for these parameters may change based on the transducer module design used. Once the cell population parameters have been identified as relevant, determining a suitable reference range for a particular analyzer configuration is routine. Using two or more of these criteria may increase the sensitivity and/or specificity of the cellular analysis for sepsis prediction.

The cell population parameters described herein, alone or in combination with other cellular analyses, may be used in conjunction with current standard of care, such as qSOFA and physical examination by a clinician (looking, e.g., for fever, altered mental state, tachycardia, tachypnea, hypotension, or other symptoms that may be undetectable or unreliably detectable from cellular analysis, blood chemistry, immunoassay, or other laboratory tests). Using the “Sepsis-2” consensus definition, the standard of care would include assessment of the patient for SIRS. A patient is considered to have SIRS when two or more of the following criteria are met: a temperature greater than 38 degrees Celsius (C) or less than 36 degrees C., a heart rate greater than 90 beats per minute (bpm), or a respiratory rate greater than 20 breaths per minute (breaths/min) and a white blood cell count (WBC) less than 4,000 per microliter of blood (4,000/mm³) (leukopenia) or greater than 12,000/mm³ (leukocytosis). Under Sepsis-2, a patient is considered septic if the patient has at a minimum of 2 SIRS plus a persistent infection (bacterial, viral or fungal) or suspicion of infection. Using the “Sepsis-3” consensus definition, the standard of care would include a Sequential Organ Failure Assessment (SOFA) or a Quick SOFA (qSOFA). A qSOFA is scored on a scale from 0-3 points, with 1 point for each symptom that tests positively. These symptoms are a respiratory rate of over 22 breaths/min, systolic arterial blood pressure of less than or equal to 100 mmHg, and an altered mental status. It has been determined that patients with a qSOFA score of at least 2 have a 24% in-hospital mortality rate, and 3% for patients with a qSOFA score of less than 2. Typically, if the qSOFA score is of at least 2, then the patent will be evaluated with the full SOFA test. The SOFA test is scored on a scale of 0-24 and involves evaluating specific organ systems (respiratory, cardiovascular, liver, renal, coagulation, and central nervous system). If the SOFA score is also greater than or equal to 2, then the patient is considered septic under Sepsis-3. In some aspects a single cell population parameter may be used with or without other cellular analysis parameters, such as WBC and/or MDW, to provide additional insight into a patient's sepsis status when the standard of care does not result in a clear diagnosis. For example, under the Sepsis-2 criteria, an inability to definitively identify an infection, e.g., using blood culture, or an inability to wait for testing to confirm infection, e.g., if the patient's condition is too tenuous to wait several days for blood culture results, one or more cell population parameters with minimally acceptable specificity and sensitivity, such as a specificity and sensitivity greater than 70%, or greater than 80%, or greater than 90%, may give a clinician confidence in initiating prophylactic treatment or heightened patient monitoring (such as in-patient admission for professional monitoring) by affirming other indicators of sepsis or developing sepsis.

The dimensions and values disclosed herein are not to be understood as being strictly limited to the exact numerical values recited. Instead, unless otherwise specified, each such dimension is intended to mean both the recited value and a functionally equivalent range surrounding that value to include at least the variability due to the reproducibility of measurements made using the test methods described herein, or industry-standard test methods if no test method is expressly disclosed.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

When used in the claims, the phrase “means for detecting whether a differentially expressed sepsis cell population parameter is present in a heterogenous population of circulating cells and characterizing an inflammatory response to infection based at least in part on that detection” should be understood as a means plus function limitation as provided for in 35 U.S.C. § 112(f), in which the functions “detecting whether a differentially expressed sepsis cell population parameter is present in a heterogenous population of circulating cells” and “characterizing an inflammatory response to infection based at least in part on that detection” are both recited, in which the corresponding structure for the first function is a computer configured to perform acts as illustrated with reference numbers 710-720 and described in the corresponding text, and the corresponding structure for the second function is a computer configured to perform acts as illustrated with reference numbers 725-730 and described in the corresponding text.

When used in the claims, the phrase “means for measuring RF conductivity” should be understood as a means plus function limitation as provided for in 35 U.S.C. § 112(f), in which the function is measuring RF conductivity, and the corresponding structure is electrodes as illustrated with reference numbers 334 and 336 and described in the corresponding text.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for characterizing inflammatory response to infection, the method comprising:
a. flowing a body fluid sample through a flowcell, the body fluid sample comprising a heterogenous population of circulating cells;
b. detecting whether a differentially expressed sepsis cell population parameter is present in the heterogenous population of circulating cells by analyzing one or more cell population parameters selected from the group consisting of MO_DC_SD, NE_DC_SD, NE_DC_MEAN, NNRBC_UMALS_SD, MO_ALL_SD, NE_NO, LY_PC, NNRBC_MALS_SD, WNOP, MO_DC_MEAN, WDOP, NNRBC_UMALS_MEAN, BA_PC, NNRBC_MALS_MEAN, BA_PC, NNRBC_MALS_MEAN, EGC_LMALS_MEAN, NNRBC_DC_SD, NNRBC_LMALS_SD, NNRBC_ALL_MEAN, and combinations thereof based on light scatter and/or direct current impedance measurements of the body fluid sample; and
c. characterizing an inflammatory response to infection as abnormal based at least in part on detecting the differentially expressed sepsis cell population parameter.

2. The method of claim 1 wherein the body fluid sample is whole blood.

3. The method of claim 2 wherein the one or more cell population parameters is analyzed for cells from the plurality of cells classified as NNRBC.

4. The method of claim 1, wherein each analyzed cell population parameter is compared to a corresponding reference range.

5. The method of claim 4 wherein the inflammatory response to infection is characterized as abnormal if at least one of the analyzed cell population parameters is outside its corresponding reference range.

6. The method of claim 4, wherein the inflammatory response to infection is characterized as abnormal if all of the analyzed cell population parameters are outside their corresponding reference ranges.

7. The method of claim 5, further comprising determining whether the distribution width of measured volumes for a population of monocytes (MDW) within the body fluid sample is within an MDW reference range.

8. The method of claim 7, wherein the inflammatory response to infection is characterized as abnormal if at least one of the analyzed cell population parameters is outside its corresponding reference range and the distribution width of the volume of the monocytes is greater than 19 channels.

9. The method of claim 5 further comprising determining whether a count of white blood cells (WBC) in the body fluid sample is within a normal reference range.

10. The method of claim 9, wherein the inflammatory response to infection is characterized as abnormal if at least one of the analyzed cell population parameters is outside its corresponding reference range and the WBC is less than 4,000 cells/mm³ or greater than 12,000 cells/mm³.

11. A system for characterizing inflammatory response to infection, the system comprising:
a. a transducer module configured to measure at least light scatter and direct current impedance caused by cells passing through a flowcell; and
b. a processor configured with a set of instructions stored on a non-transitory computer readable medium and operable to, when executed:
i. detect whether a differentially expressed sepsis cell population parameter is present in a heterogenous population of circulating cells by analyzing one or more cell population parameters selected form the group consisting of MO_DC_SD, NE_DC_SD, NE_DC_MEAN, NNRBC_UMALS_SD, MO_ALL_SD, NE_NO, LY_PC, NNRBC_MALS_SD, WNOP, MO_DC_MEAN, WDOP, NNRBC_UMALS_MEAN, BA_PC, NNRBC_MALS_MEAN, BA_PC, NNRBC_MALS_MEAN, EGC_LMALS_MEAN, NNRBC_DC_SD, NNRBC_LMALS_SD, NNRBC_ALL_MEAN, and combinations thereof based at least in part on the light scatter or direct current impedance measurements; and
ii. characterize an inflammatory response to infection based at least in part on the one or more cell population parameters.

12. The system of claim 11, wherein the processor is further configured to compare at least one of the analyzed cell population parameters to a corresponding reference range.

13. The system of claim 12, wherein the processor is configured to characterize the inflammatory response to infection as abnormal if the at least one of the analyzed cell population parameters is outside the corresponding reference range.

14. The system of claim 12, wherein the processor is further configured to identify monocytes and measure the volume of monocytes among the cells passing through the flowcell and calculate a distribution width of the monocyte volume measurements (MDW).

15. The system of claim 14, wherein the processor is further configured to compare the MDW to an MDW reference range, and to characterize the inflammatory response to infection as abnormal if the at least one of the analyzed cell population parameters is outside its corresponding reference range and the MDW is outside the MDW reference range.

16. The system of claim 12, wherein the processor is further configured to identify and count white blood cells (WBC) among the cells passing through the flow cell.

17. The system of claim 16, wherein the processor is further configured to:
a. compare the at least one of the analyzed cell population parameters to its corresponding reference range;
b. compare the MDW to an MDW reference range;
c. compare the WBC to a WBC reference range; and
d. characterize the inflammatory response to infection based on a combination of at least these comparisons.

18. The system of claim 17, wherein the inflammatory response to infection is characterized as abnormal if the at least one of the analyzed cell population parameters is outside its corresponding reference range, the MDW is outside the MDW reference range and the WBC is outside the WBC reference range.

19. The system of claim 17, wherein local decision rules are applied to characterize the inflammatory response to infection if the at least one of the analyzed cell population parameters, the MDW and the WBC are not all within or all outside of their respective reference ranges.

20. A machine comprising:
a. a transducer module configured to measure at least light scatter and direct current impedance caused by cells passing through a flowcell, wherein the transducer module comprises means for measuring RF conductivity, wherein the means for measuring RF conductivity is operable to measure RF conductivity of cells passing through the flowcell and is also operable to measure RF conductivity of cells passing through a second flowcell comprised by the transducer module; and
b. means for detecting whether a differentially expressed sepsis cell population parameter is present in a heterogenous population of circulating cells and characterizing an inflammatory response to infection based at least in part on that detection.
